SEQUENCE LISTING (1 sequence)

SEQ ID NO: 1 - RNA, 24 nt, Artificial Sequence
  synthetic
  misc_feature (18)..(18): 3-methyl-6-amino-5-(1′-beta-D-ribofuranosyl)- pyrimidin-2-one
gggaguguuguauuuggncaauuu

(19) United States
(12) Patent Application Publication
(10) Pub. No.: US 2021/0238643 A1
(43) Pub. Date: Aug. 5, 2021

(54) Enzymatic Processes for Synthesizing RNA Containing Certain Non-Standard Nucleotides

(71) Applicants: Nicole A Leal, Gainesville, FL (US); Steven A Benner, Gainesville, FL (US)

(72) Inventors: Nicole A Leal, Gainesville, FL (US); Steven A Benner, Gainesville, FL (US)

(21) Appl. No.: 17/188,248

(22) Filed: Mar. 1, 2021

Related U.S. Application Data
(63) Continuation-in-part of application No. 16/226,963, filed on Dec. 20, 2018, now Pat. No. 10,934,569.

(51) Int. Cl. C12P 19/34; C07H 21/02

(52) U.S. Cl. CPC C12P 19/34; C07H 21/02

(57) ABSTRACT

This invention relates to nucleotide analogs and their derivatives (termed non-standard nucleotides) that, when incorporated into DNA and RNA, expand the number of nucleotides beyond the four found in standard DNA and RNA. The invention further relates to enzymatic processes that incorporate those non-standard nucleotide analogs into oligonucleotide products using the corresponding triphosphate derivatives. The RNA polymerases of the instant invention transcribe DNA containing nonstandard nucleotides to give RNA containing nonstandard nucleotides, where certain of those nucleotides have nucleobases that do not present electron density to the minor groove.

### CROSS REFERENCE TO RELATED APPLICATIONS

This application is a continuation in part of U.S. patent application Ser. No. 16/226,963, currently copending, filed 20 Dec. 2018, for “Enzymatic Processes for Synthesizing RNA Containing Certain Non-Standard Nucleotides”.

### STATEMENT OF RIGHTS TO INVENTIONS MADE UNDER FEDERALLY-SPONSORED RESEARCH

This invention was made with government support under grants from the National Institutes of Health (R01GM128186). The government has certain rights in the invention.

### THE NAMES OF PARTIES TO A JOINT RESEARCH AGREEMENT

Not applicable

### INCORPORATION BY REFERENCE OF MATERIAL SUBMITTED ON A COMPACT DISK

None

### BACKGROUND OF THE INVENTION

### 1. Field of the Invention

This invention relates to nucleotide analogs and their derivatives (termed non-standard nucleotides) that, when incorporated into DNA and RNA, expand the number of nucleotides beyond the four found in standard DNA and RNA. The invention further relates to enzymatic processes that incorporate those non-standard nucleotide analogs into oligonucleotide products using the corresponding triphosphate derivatives. The RNA polymerases of the instant invention transcribe DNA containing nonstandard nucleotides to give RNA containing nonstandard nucleotides, where certain of those nucleotides have nucleobases that do not present electron density to the minor groove.

### 2. Description of the Related Art

Natural oligonucleotides bind to complementary oligonucleotides according to rules of nucleobase pairing first elaborated by Watson and Crick in 1953, where adenine (A) pairs with thymine (T) (or uracil, U, in RNA), and guanine (G) pairs with cytosine (C), with the complementary strands anti-parallel to each other. These rules arise from two principles of complementarity, size-complementarity (large purines pair with small pyrimidines) and hydrogen bonding complementarity (hydrogen bond donors pair with hydrogen bond acceptors).

It is now well established in the art that the number of independently replicable nucleotides in DNA can be increased, where the size- and hydrogen binding complementarities are retained, but where different heterocycles (nucleobases or, as appropriate, nucleobase analogs) attached to the sugar-phosphate backbone implement different hydrogen bonding patterns. As many as eight different hydrogen bonding patterns forming four additional nucleobase pairs are conceivable (see, for example, [Benner, S. A. (1995) Non-standard Base Pairs with Novel Hydrogen Bonding Patterns. U.S. Pat. No. 5,432,272 (Jul. 11, 1995)]). This has led to an “artificially expanded genetic information system” (AEGIS). As illustrated in FIG. 1, different nucleobases/nucleobase analogs/heterocycles can implement the same hydrogen bonding pattern, standard or non-standard.

Additional nucleobase pairs have had substantial use in diagnostics, in part because the alternative hydrogen bonding patterns support orthogonal pairing. There and in this disclosure, “DNA” includes oligonucleotides containing nucleic acids and their analogs carrying tags (e.g., fluorescent, functionalized, or binding) to the ends, sugars, or nucleobases.

It would also be useful to transcribe DNA oligonucleotides containing non-standard components to give RNA containing complementary non-standard components. For example, messenger RNA containing non-standard components and transfer RNA containing the complementary non-standard components, may be used in ribosome-mediated translation to incorporate non-standard amino acids into a peptide [Bain, J. D., Chamberlin, A. R., Switzer, C. Y., Benner, S. A. (1992) Ribosome-mediated incorporation of non-standard amino acids into a peptide through expansion of the genetic code. Nature 356, 537-539].

Indeed, the art contains descriptions of procedures that do transcribe DNA oligonucleotides containing AEGIS components to give RNA containing complementary non-standard components [Leal, N. A., Kim, H.-J., Hoshika, S., Kim, M.-J., Carrigan, M. A., Benner, S. A. (2015) Transcription, reverse transcription, and analysis of RNA containing artificial genetic components. ACS Synthetic Biol. 4, 407-413]. However, without wishing to be bound by theory, for transcription to be successful, it appears that the non-standard components must not differ from standard nucleotide components in one critical way: They must present electron density into the minor groove, either from the nitrogen at position 3 analogous to N3 of standard purines, or from the exocyclic oxygen from the C═O group at position 2 analogous to the 2-position C═O of cytosine and thymine/uracil.

Theory notwithstanding, the art reports examples where a nonstandard ribonucleoside triphosphate that is an analog of a pyrimidine that presents, instead of a C═O group and its electron density, an —NH2 group at the position analogous to the 2-position, fails to be incorporated into RNA by enzymatic transcription of a DNA template containing the corresponding nonstandard templating nucleotide [C. Y. Switzer, S. E. Moroney, S. A. Benner, Enzymatic recognition of the base pair between iso-cytidine and iso-guanosine. Biochemistry 32, 10489-10496 (1993)]. For this reason, the art does not enable this kind of transcription, especially when the pyrimidine analog is isocytidine or its analogs (e.g. pseudocytidine), diaminopyrimidine, 2,4-diaminopyridine or its derivatives (e.g., the 5-nitro derivative), 2-aminopyridin-4-ones and their derivatives (e.g., the 5 nitro derivative), and purine derivatives such as xanthosine and 7-deazaxanthosine that have an NH at the 3-position in the purine numbering scheme (FIG. 1). Processes that perform this transcription are the goal of this invention.

### BRIEF SUMMARY OF THE INVENTION

This invention covers processes for transcribing DNA oligonucleotides to give RNA transcripts that incorporate non-standard nucleotides that do not present electron density to the minor groove. Those processes depend on variants of RNA polymerases that accept nonstandard nucleotides that do not present electron density to the minor groove. Further described for the first time is a DNA-like system that has eight different nucleotide-like building blocks with predictable pairing. Inventive parameters are provided that allow useful prediction of the pairing of duplexes containing certain standard and non-standard nucleobase pairs.

### BRIEF DESCRIPTION OF THE DRAWINGS

FIG. 1. Non-standard nucleotides of the instant invention. Where Q=C—H (carbon-hydrogen), or C-M (carbon-M), or N, where M is an alkyl, alkenyl, or alkynyl substituent, either simple or functionalized. Note how the four standard nucleotides (labeled G, A, C, and T) all deliver electron density to the minor groove from their purine N7 or the exocyclic oxygen of the purines. Note how the heterocycle of the non-standard pyrimidine analog labeled Z also does so, but that the pyrimidine analogs labeled S, K, and V do not, nor does the implementation of the X hydrogen bonding pattern with a “Q” at position 7.

FIG. 2. The presently preferred nucleotides of the instant invention.

FIG. 3. A plot showing experiments and predictions for the 8-letter system of the instant invention. Plot of experimental vs. predicted free energy changes (ΔG^{o}₃₇) for 94 SBZP-containing 8-letter DNA duplexes.

FIG. 4. Plot of experimental vs. predicted melting temperatures of 94 SBZP-containing 8-letter DNA duplexes in this study (data in Tables 3, 6, and 8).

FIG. 5. Schematic showing an analog of a fluorescent aptamer known in the art as “spinach”, with non-standard ribonucleotides Z, B, S, and P.

FIG. 6. Fluorescence of the 8-letter spinach construct. From left to right: (a) native spinach aptamer with fluor, (b) fluor and spinach aptamer containing Z at position 50, near the fluor, which binds in L12, (c) Control with fluor only, lacking RNA, and (d) full 8-letter spinach having the sequence shown in the left panel. Images are created under 400 nm light with an orange filter.

FIG. 7. Plot comparing the experimental free energy changes, ΔG^{o}₃₇, with the free energy changes predicted from the parameters determined here for the eight-letter DNA analog of the instant invention. These were generated for duplexes in this study (data in Table 3 and 4). NN parameters and standard errors, sigma, for Z-P containing NN dimers were derived by SVD and standard error propagation.

FIG. 8. Plot comparing the experimental melting temperatures vs. the predicted Tm's for 41 Z-P containing DNA duplexes (data in Table 3 and 4). All Tm's were calculated using a total oligonucleotide concentration of 1×10⁻⁴ M.

FIG. 9. Plot comparing the experimental free energy changes, ΔG^{o}₃₇, versus the predicted free energy changes for all 37 duplexes in this study (data in Table 6 and 7). NN parameters and standard errors, sigma, for S-B containing NN dimers were derived by SVD and standard error propagation.

FIG. 10. Plot of the experimental melting temperatures vs. the predicted melting temperatures for all 37 S-B containing DNA duplexes (data in Table 6 and 7). All Tm's were calculated using a total oligonucleotide concentration of 1×10⁻⁴ M.

FIG. 11. Experimental vs. predicted free energies of SBZP-containing 8-letter DNA duplexes. Plotted are experimental free energy changes (ΔG^{o}₃₇) versus predicted free energy changes for all 15 duplexes in this study (data in Tables 9 and 10). Parameters for dinucleotide pairing affinity and standard errors, sigma, for dinucleotides containing P and Z dimers were derived by singular value decomposition and standard error propagation.

FIG. 12. Plot of experimental melting temperatures vs. predicted melting temperatures for all 15 S-B and Z-P dinucleotides in DNA duplexes (data in Table 9 and 10). All Tm's were calculated using a total oligonucleotide concentration of 1×10⁻⁴ M.

FIG. 13. PAGE (20%) showing transcription products with internal labeling. Wild type and mutant T7 RNA polymerases were tested in the absence and presence of rSTP for their ability to generate the RNA product T2S; they show different levels of pausing and rescue. Full length product is a 24mer, S is at position 18; pausing is most prominent at position 17. T7, the FA variant (with Y639F and H784A replacements, the “FL variant”) and the FAL variant (with Y639F, H784A, and P266L replacements, the “FAL variant”) show pausing in the absence of riboSTP and various levels of rescue in the presence of riboSTP. The experimental data are collected in clusters of four sequences with the top cluster being the variant of T7 RNA polymerase (native, F, FL, FA, FAL (A), VRS, and FAL (B), the last at lower concentration), each having two lanes without riboSTP, and two lanes with riboSTP, with incubation times of 2 and 16 hours. The variants are defined as Y639F, Y639F P266L,

FIG. 14. HPLC (ammonium bicarbonate, 0 to 200 mM) traces of the rN-3′-monophosphates recovered by RNase T2 digestion of the RNA made by attempts with different RNA polymerase variants to make 8-letter spinach. (Left) Trace from RNA made via transcription using wild-type T7 RNA polymerase. Note absence of S-3′-P. (Center) Trace from RNA made via transcription using the FAL variant of T7 RNA polymerase. Note detectable presence of S-3′-P, notwithstanding its low extinction coefficient and its expected presence in the transcript as only one exemplar. (Right). Trace from RNA made via transcription using the FAL variant T7 RNA polymerase, with co-injection of the authentic rS-3′-monophosphate made by chemical synthesis. The expected 8-letter transcript is:

GGG AGU GUU GUA UUU GGS CAA UUU SEQ ID 1

with one S relative to 5 {A+C}, 8 G, and 10 U. Using the extinction coefficients above, 1.2±0.4 S nucleotides were incorporated into the transcript by the FAL variant of T7 RNA polymerase.

FIG. 15 A-FIG. 15 C. HPLC trace (ammonium bicarbonate, 0 to 200 mM) of rN-3′-monophosphates recovered by RNase T2 digestion of the spinach aptamer made by transcription of an 8-letter template. (15 A) Products from the aptamer made by wild-type T7 RNA polymerase; it does not contain S-3′-P, as confirmed by TLC. (15 B) Products from the aptamer made by the FAL variant of T7 RNA polymerase containing all eight components (G, A, C, T, Z, P, S, and B). (15 C) Products from the aptamer made by the FAL variant of T7 RNA polymerase with co-injection of the authentic rZ-3′-monophosphate made by chemical synthesis.

FIG. 16 2D-TLC of RNase T2 digests of labeled test sequences (panels A-D) and spinach (panels E and F) made with wild-type T7 RNA polymerase in primary solvent system. (A) With template giving a product containing P as the only 8-letter non-standard nucleotide, generates P-3′-′²P (Pp) after digestion. (B) With template giving a product containing Z as the only 8-letter non-standard nucleotide, generates Z-3′³²P (Zp) after digestion, which runs with U-3′-P. (C) With template that produces a product containing S as the only 8-letter non-standard nucleotide, wild-type T7 RNA polymerase apparently does not incorporate STP (absence of S-3′-³²P which would run to the right of C-3′-P in this solvent system, shown below). (D) With template giving a product containing B as the only 8-letter non-standard nucleotide, generates B-3′³²P (Bp) after digestion. (E) Transcript of the spinach aptamer using alpha-³²P-GTP, which nearest neighbor labels all four standard nucleotides, as well as Z and P. After digestion, evidence of incorporation comes from the appearance of the corresponding Z-3′³²P (Zp) and P-3′³²P (Pp). Since Z-3′-P does not separate convincingly in this system, its presence in the spinach aptamer was confirmed by HPLC (Figure E10), and in a second buffer system (shown below). (F) Transcript of the spinach aptamer using alpha-³²P-CTP, which nearest neighbor labels all four standard nucleotides, as well as S and B. After digestion, evidence of incorporation of BTP comes from the appearance of the corresponding B-3′³²P (Bp). However, essentially no amount of radioactivity is attributable to S-3′³²P. This suggests the need to use a variant of T7 RNA polymerase to allow the preparation of 8-letter RNA from 8-letter DNA by transcription. In addition, a secondary TLC system was required to resolve all eight 3′-phosphates arising from all eight components of the 8-letter system.

FIG. 17 2D-TLC of RNase T2 digests of labeled test sequences (panels A-D) and spinach (panels E and F) made with wild-type T7 RNA polymerase in secondary solvent system. (A) With template giving a product containing P as the only 8-letter non-standard nucleotide, generates P-3′³²P (Pp) after digestion. (B) With template giving a product containing Z as the only 8-letter non-standard nucleotide, generates Z-3′³²P (Zp) after digestion, which now runs much slower, separate from U-3′P. (C) With template that produces a product containing S as the only 8-letter non-standard nucleotide, essentially no amount of radioactivity is attributable to S-3′-³²P. (D) With template giving a product containing B as the only 8-letter non-standard nucleotide, generates B-3′-³²P (Bp) after digestion. (E) Transcript of the spinach aptamer using alpha-³²P-GTP, which nearest neighbor labels all four standard nucleotides, as well as Z and P. After digestion, evidence of incorporation comes from the appearance of the corresponding Z-3′-³²1³ (Zp) and P-3′-³²P (Pp). (F) Transcript of the spinach aptamer using alpha-³²P-CTP, which nearest neighbor labels all four standard nucleotides, as well as S and B. After digestion, evidence of incorporation of BTP comes from the appearance of the corresponding B-3′-³²P (Bp). However, essentially no radioactivity is attributable to S-3′-³²P. This again suggests the need to use a variant of T7 RNA polymerase to allow the preparation of 8-letter RNA from 8-letter DNA by transcription.

FIG. 18 2D-TLC of RNase T2 digests of labeled test sequences (panels A-D) and spinach (panels E and F) made with FAL variant of T7 RNA polymerase in primary solvent system. (A) With template giving a product containing P as the only 8-letter non-standard nucleotide, generates P-3′-³²P (Pp) after digestion. (B) With template giving a product containing Z as the only 8-letter non-standard nucleotide, generates Z-3′-³²P (Zp) after digestion, which runs with U-3′-P. (C) With template that produces a product containing S as the only 8-letter non-standard nucleotide, S-3′-³²P is now clearly present. (D) With template giving a product containing B as the only 8-letter non-standard nucleotide, generates B-3′-³²P (Bp) after digestion. (E) Transcript of the spinach aptamer using alpha-³²P-GTP, which nearest neighbor labels all four standard nucleotides, as well as Z and P. After digestion, evidence of incorporation comes from the appearance of the corresponding Z-3′-³²P (Zp) and P-3′-³²P (Pp). Since Z-3′-P does not separate convincingly in this system, its presence in the spinach aptamer was confirmed by HPLC (Figure E10). (F) With the spinach aptamer labeled with C-alpha-³²P-triphosphate, label of G, A, C, U, S, and B is expected. All six spots are seen in the amounts approximately as expected.

FIG. 19 2D-TLC of RNase T2 digests of labeled test sequences (panels A-D) and spinach (panels E and F) made with FAL variant of T7 RNA polymerase in secondary solvent system. (A) With template giving a product containing P as the only 8-letter non-standard nucleotide, generates P-3′-³²P (Pp) after digestion. (B) With template giving a product containing Z as the only 8-letter non-standard nucleotide, generates Z-3′-³²P (Zp) after digestion. (C) With template that produces a product containing S as the only 8-letter non-standard nucleotide, S-³²P-phosphate is again clearly present. (D) With template giving a product containing B as the only 8-letter non-standard nucleotide, generates B-3′-′²P (Bp) after digestion. (E) Transcript of the spinach aptamer using alpha-³²P-GTP, which nearest neighbor labels all four standard nucleotides, as well as Z and P. After digestion, evidence of incorporation comes from the appearance of the corresponding P-3′³²P (Pp). Z-3′³²P (Zp) running near G-3′-P; its incorporation was confirmed by HPLC (Figure E10). (F) With the spinach aptamer labeled with alpha-³²P-CTP, label of G, A, C, U, S, and B is expected. All six spots are seen with B-3′³²P (Bp) running above G-3′-P and S-3′-P running to the left of C-3′-P.

### DETAILED DESCRIPTION OF THE INVENTION

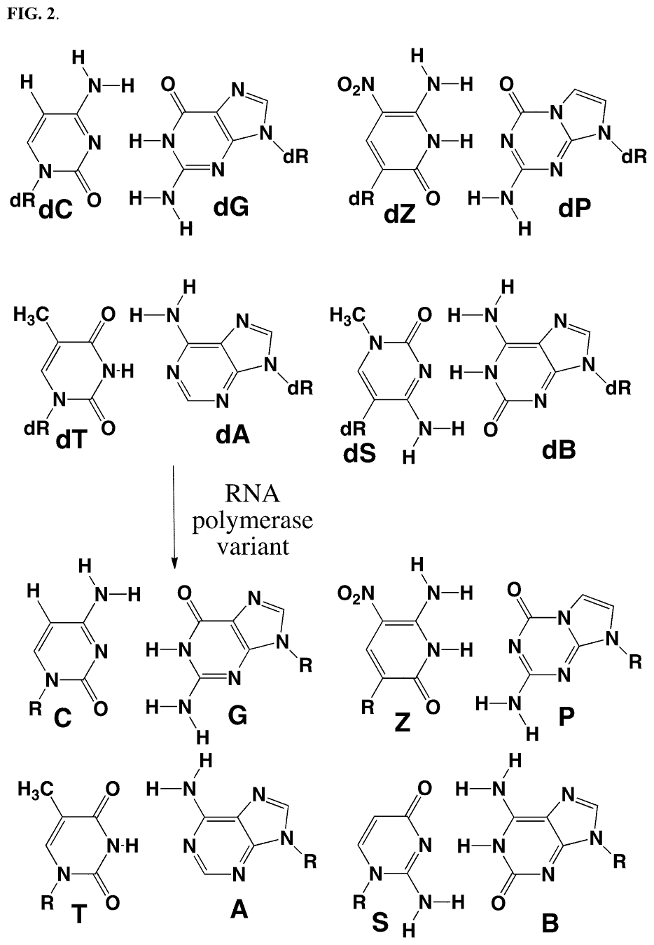

FIG. 2 shows the nucleotide nucleobases that are presently preferred in an 8-letter DNA-like system, and the nucleotide nucleobases that are presently preferred in an RNA system. To show that these eight letter systems can have utility as an information storage system, as with DNA and RNA, it must be shown that DNA analogs built from an arbitrarily large set of sequences form duplexes having predictable thermodynamic stability. This, in turn, requires determining the thermodynamic stability of an arbitrarily large number of duplexes, extracting thermodynamic binding parameters for individual pairs from them, and determining whether these yield a predictive model for the stability of duplexes. This was done following the procedure disclosed in Example 1.

With an eight-letter molecular recognition system, the number of possible dinucleotides is much larger than with just four. Considering duplex sequence symmetry, natural 4-letter DNA has ten unique base-pair dinucleotides, each with its own parameter [J. SantaLucia, Proc. Natl Acad. Sci. USA 95, 1460-1465 (1998)]. We represent these base-pair dinucleotides with a slash symbol (e.g. 5′-AC-3′ paired with 3′-TG-5′ is represented by AC/TG). These 10 dinucleotides are: AA/TT, AT/TA, TA/AT, AC/TG, AG/TC, CA/GT, GA/CT, CC/GG, GC/CG, and CG/GC J. [SantaLucia, Jr, Determination of nucleic acid thermodynamics by UV absorbance melting curves, in spectrophotometry and spectrofluorimetry: A practical approach (M. G. Gore, Ed.), Oxford U. Press (2000)]. Six other dinucleotides can be written (TT/AA, GT/CA, CT/GA, TG/AC, TC/AG, GG/CC), but due to duplex symmetry each of these is identical to one of the unique dinucleotides (e.g. AC/TG is equivalent to GT/CA). Two additional parameters improve predictions in 4-letter DNA. The first, a duplex initiation parameter, accounts for the decrease in translational degrees of freedom (an entropy penalty) when two strands become one duplex. The second parameter treats A:T pairs at the ends of duplexes specially.

A 6-letter DNA alphabet with S:B, T:A and C:G pairs adds to these 11 more NN dinucleotides, each with its own thermodynamic parameter, specifically (again considering symmetry) AS/TB, AB/TS, TS/AB, TB/AS, GS/CB, GB/CS, CS/GB, CB/GS, SS/BB, SB/BS, BS/SB. For 6-letter DNA having Z and P, 11 more NN dimers are again added, each with its own thermodynamic parameter (analogous to the SB dinucleotides given). Combining S:B and Z:P pairs in the same duplex adds four more NN dinucleotides, each with its own parameter: ZS/PB, ZB/PS, SZ/BP, and BZ/SP. Last, to get the same predictive power for 8-letter DNA as for standard DNA, 2 extra parameters are needed for S:B and Z:P pairs at the ends of duplexes. Thus, a total of 28 new parameters (i.e. unknowns) are needed; the 4-letter natural DNA code requires 12 parameters (for ten dinucleotides plus two for initiation and terminal A-T) whereas the 8-letter DNA requires 40 parameters (for 36 dinucleotides plus four for initiation with terminal G:C and terminal effects for A:T, S:B, and Z:P).

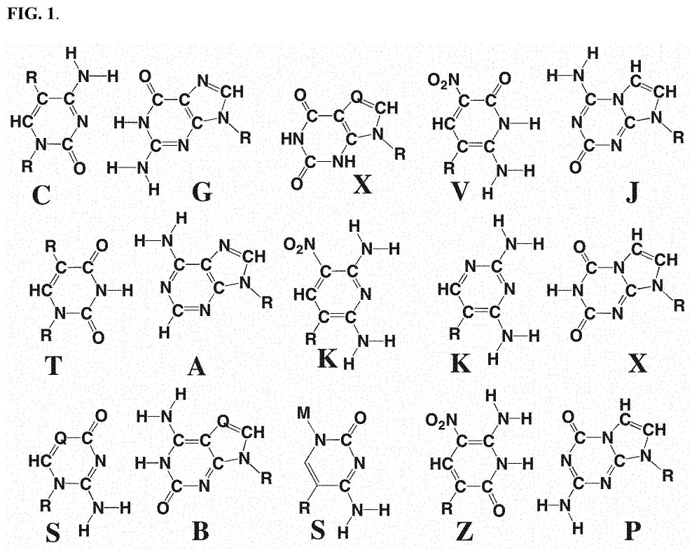

As described in Example 1, protected phosphoramidites of two additional purine nucleoside analogs “P” and “B” and two additional pyrimidine analogs “Z” and “S” (Table 1, FIG. 1) were synthesized and used in solid-phase synthesis to create 94 short oligonucleotide duplexes. These were predicted to support P:Z and B:S pairing (FIG. 1) in addition to standard G:C and A:T pairing. Thermodynamic data for these 94 duplexes were collected by measuring UV absorbance (260 nm) as a function of temperature at six different DNA concentrations in saline buffer. These conditions, often used to study standard DNA, allow direct comparison between 8-letter parameters and parameters for 4-letter DNA. Data were processed using Meltwin v.3.5 to obtain a parameter set using both the (Tm-1 vs. Ln(Ct)) method [J. SantaLucia Jr, D. H. Turner, Biopolymers 44, 309-319 (1997)] and the Marquardt non-linear curve fit method [J. SantaLucia, Jr, Determination of nucleic acid thermodynamics by UV absorbance melting curves, in spectrophotometry and spectrofluorimetry: A practical approach (M. G. Gore, Ed.), Oxford U. Press (2000)]. The error-weighted average of the values from the two methods yielded the thermodynamic values for the 94 duplexes that were used to determine the 28 new NN parameters and validate the quality of predictions [J. SantaLucia Jr, D. H. Turner, Biopolymers 44, 309-319 (1997); J. SantaLucia, Jr, Determination of nucleic acid thermodynamics by UV absorbance melting curves, in spectrophotometry and spectrofluorimetry: A practical approach (M. G. Gore, Ed.), Oxford U. Press (2000); H. T. Allawi, J. SantaLucia Jr, Biochemistry 36, 10581-10594 (1997)].

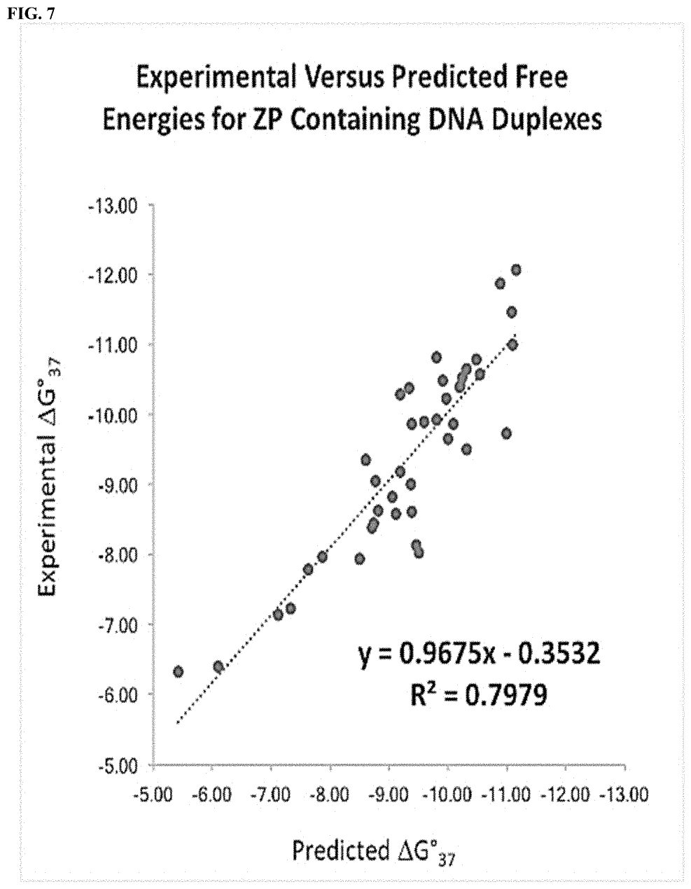

To determine the 12 new parameters involving combinations of G:C, A:T and Z:P pairs for the 6-letter GACTZP system, the duplex ΔG° 37 and ΔH° were measured for 41 duplexes (Table 4 and FIG. 7). The 12 new parameters involving combinations of G:C, A:T and B:S pairs for the 6-letter GACTBS system, the duplex ΔG° 37 and ΔH° were measured for 37 duplexes (Table 7 and FIG. 9). To determine the final 4 parameters for NN dimers with tandem B:S and Z:P pairs (i.e. ZS/PB, ZB/PS, SZ/BP, and BZ/SP) thermodynamics were measured for 15 duplexes (Table 9). FIG. 2 shows the agreement between experiments and predictions for the 8-letter system.

The thermodynamics for 94 8-letter duplexes synthesized from the 8-letter GACTSBZP DNA alphabet were then measured. These were used to obtain, and obtained best fit 28 parameters to these using singular value decomposition. Because this number of measurements over-determines these unknowns by a factor of 3.3, we were able to test the applicability of the NN model and to use error propagation to derive standard deviations in the derived parameters. The NN parameters

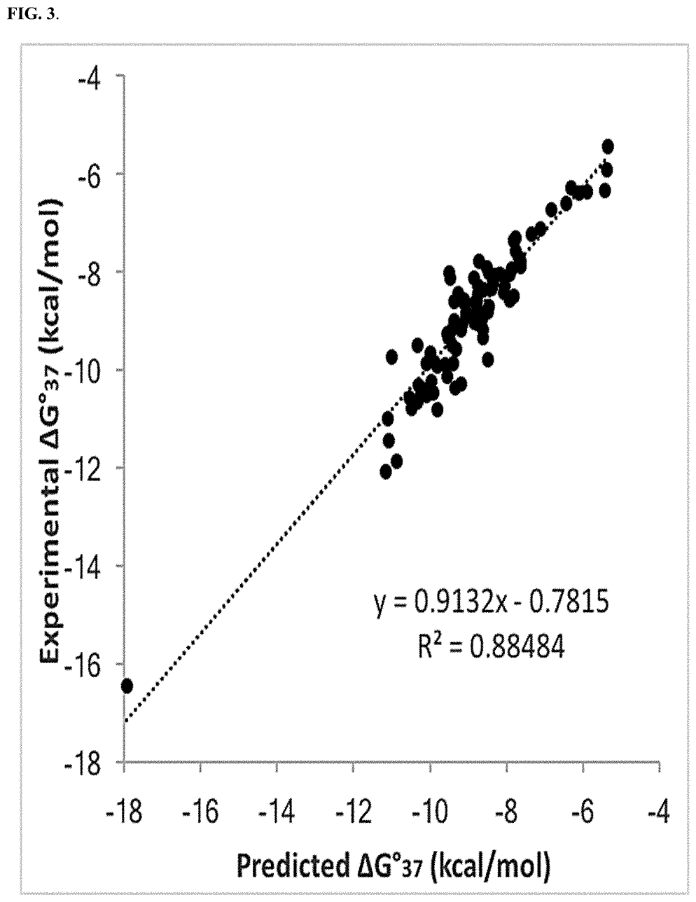

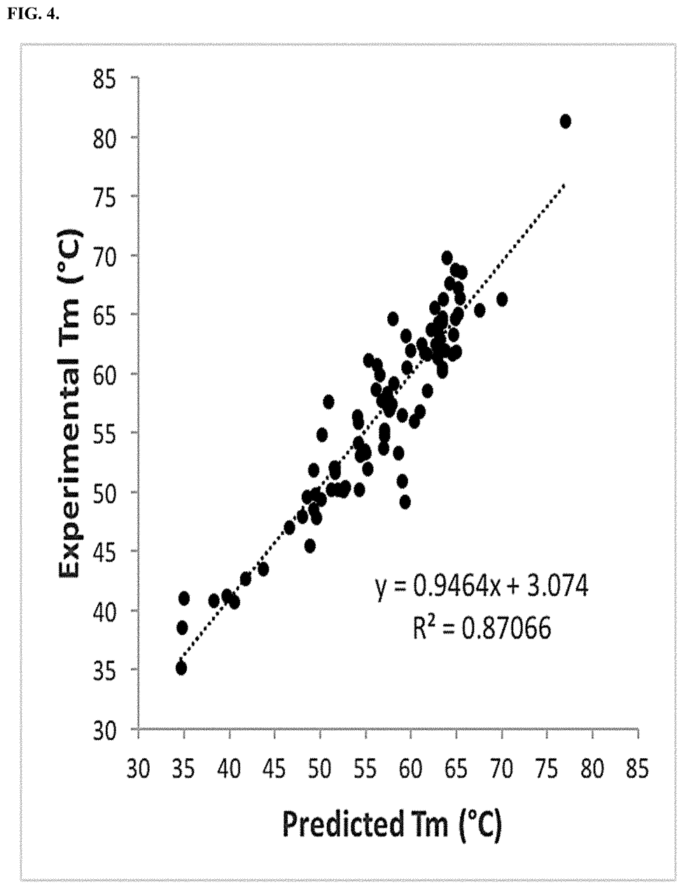

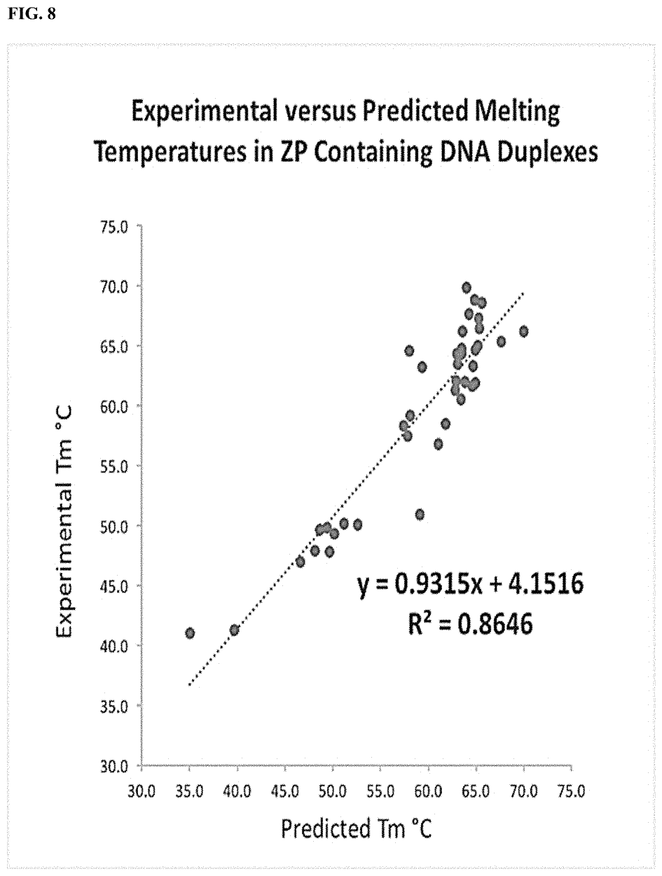

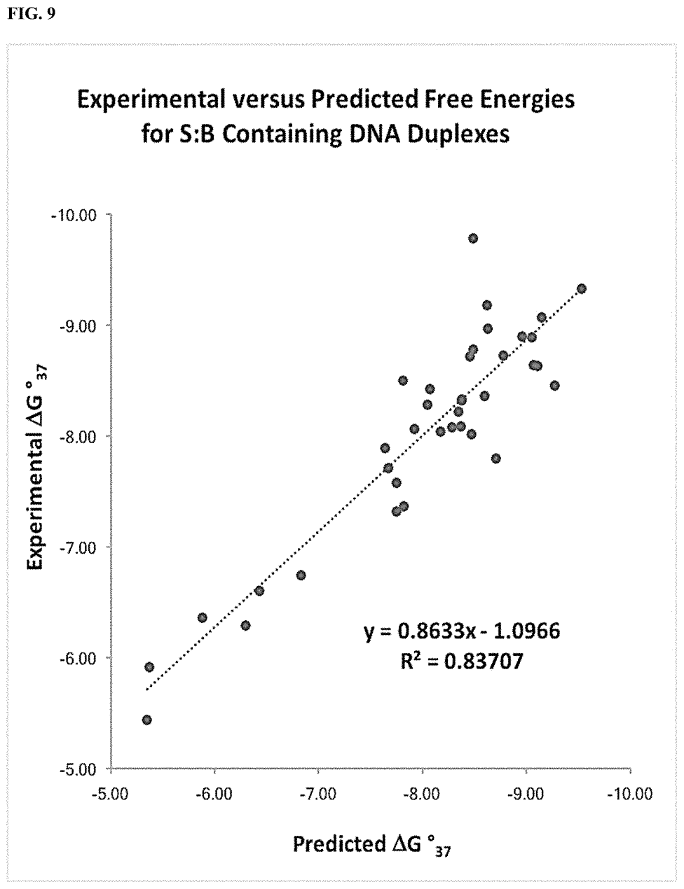

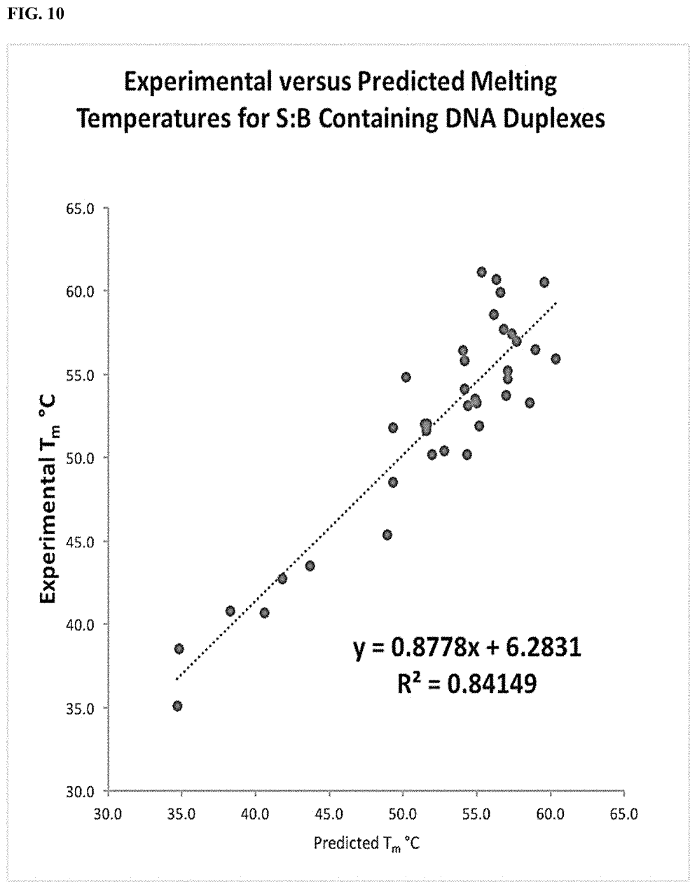

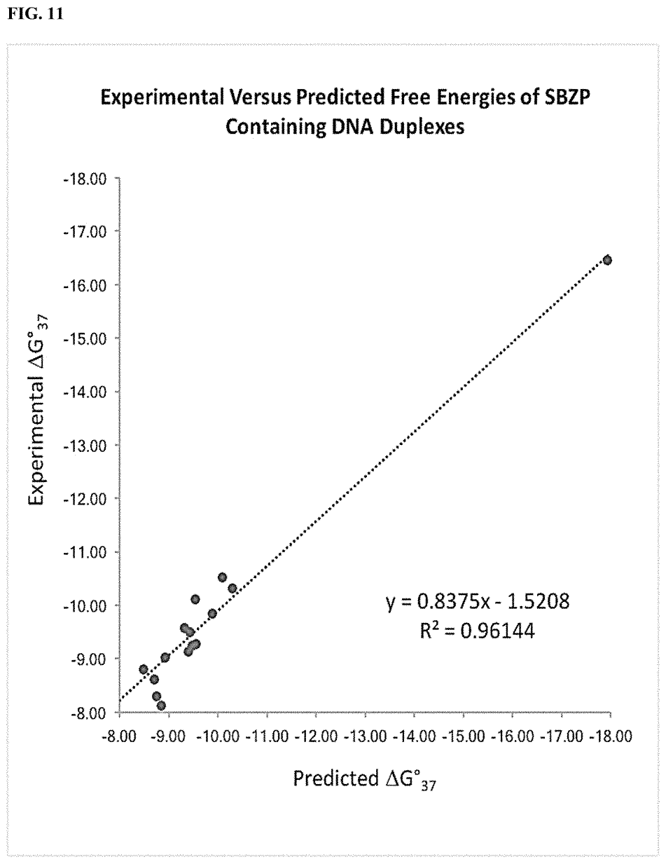

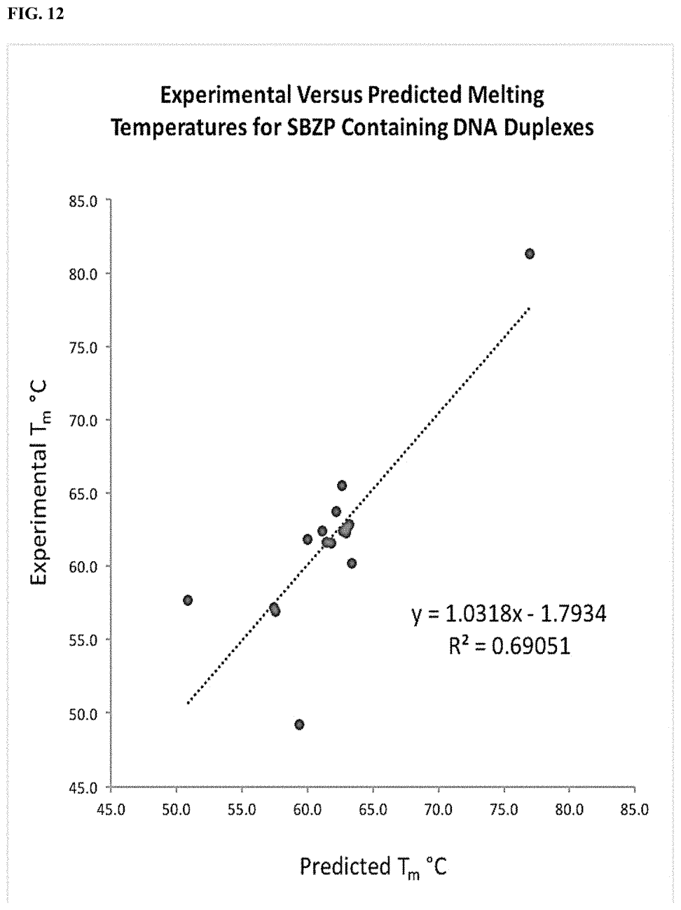

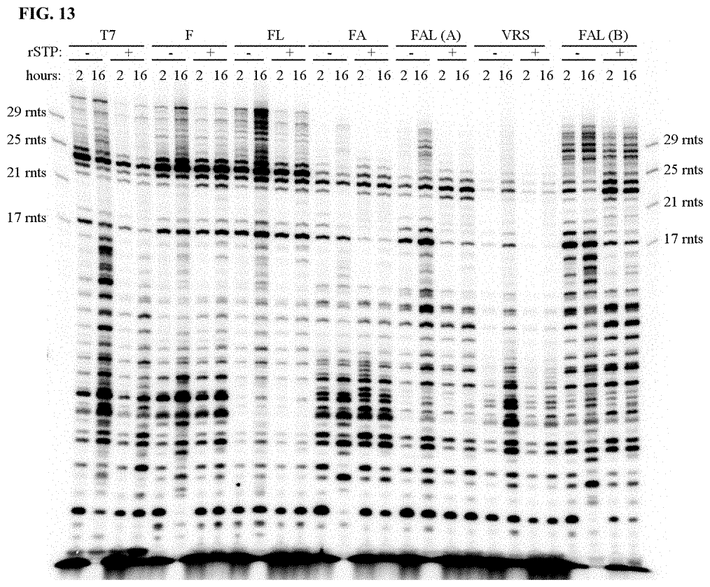

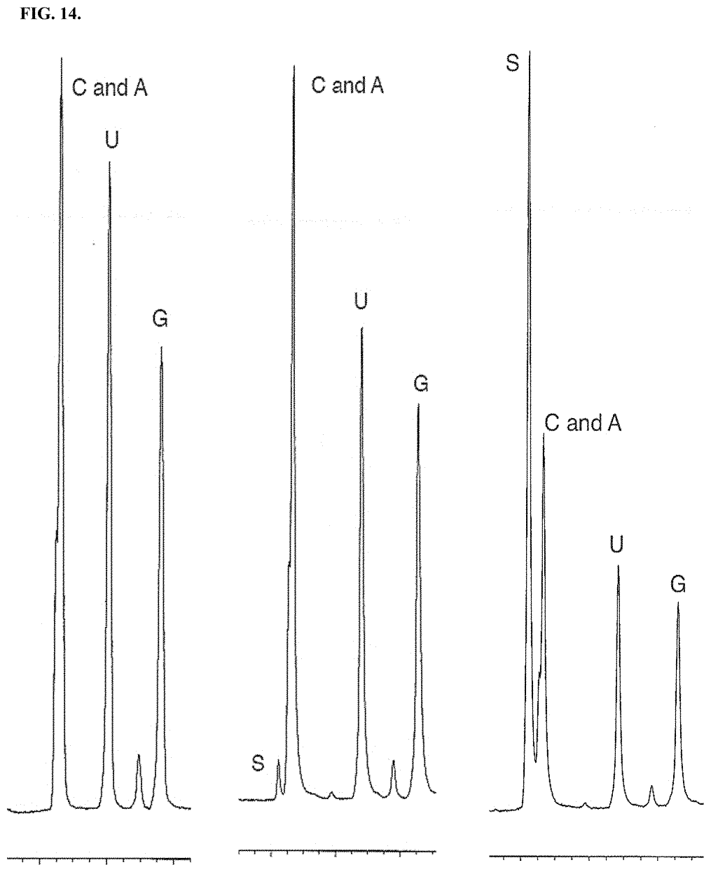

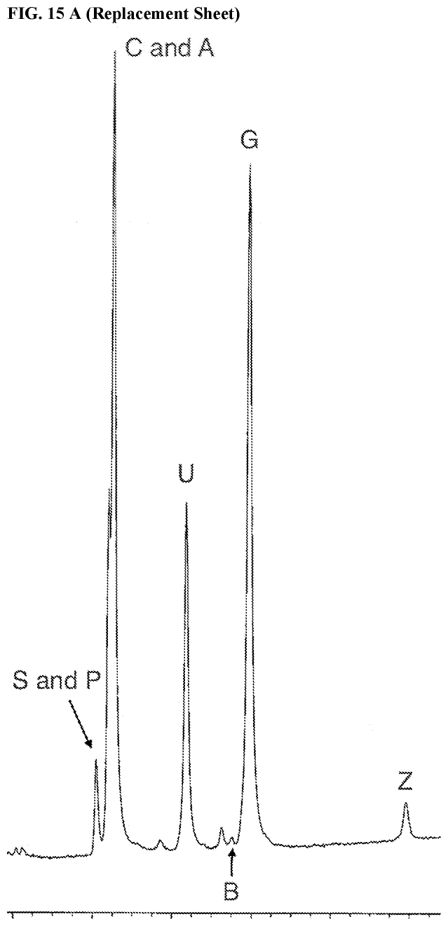

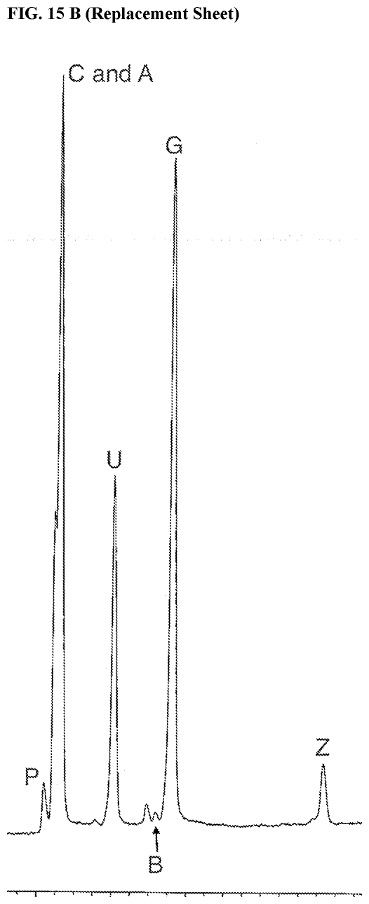

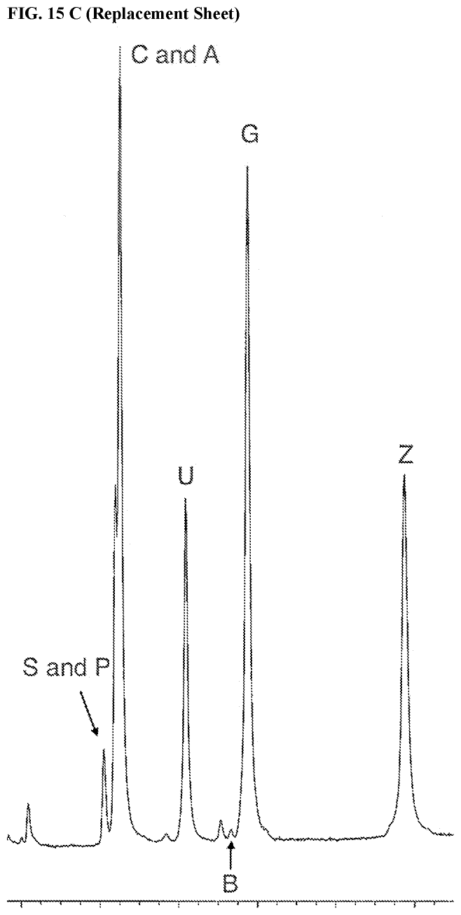

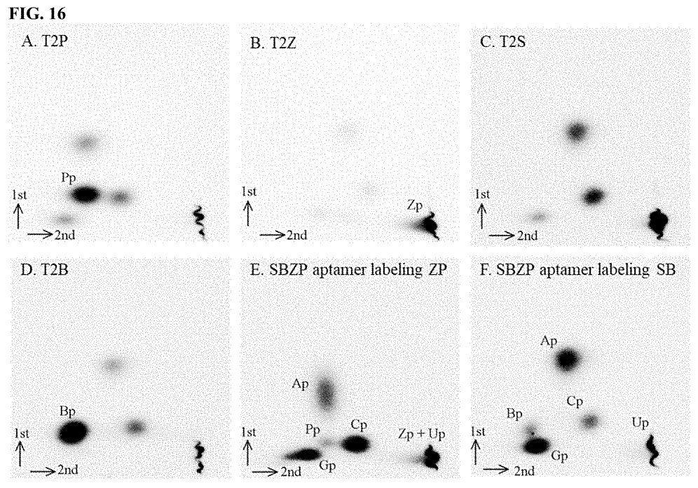

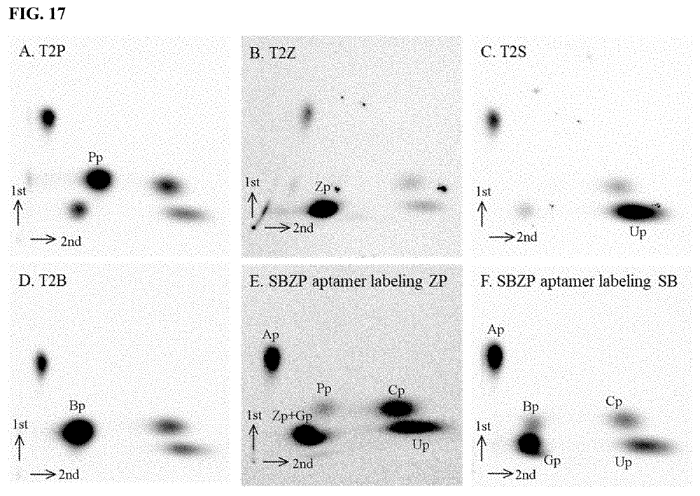

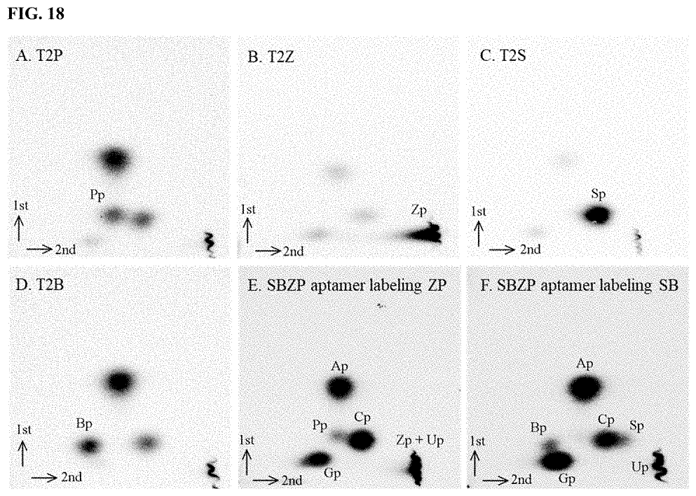

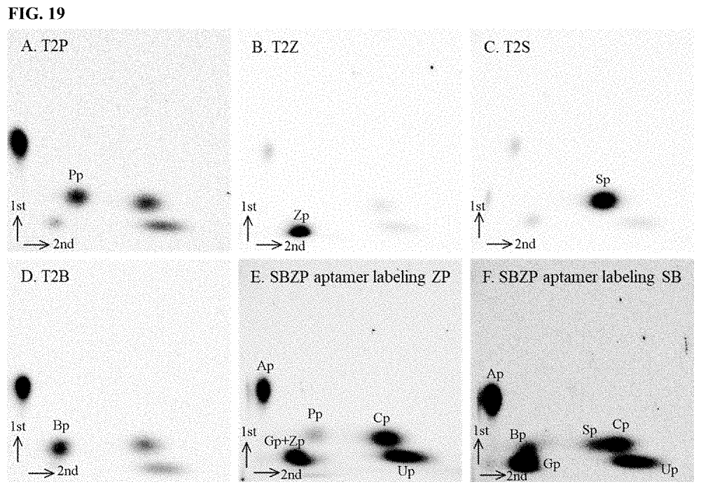

FIG. 3 shows a plot of experimental versus the predicted free-energy changes based on software that incorporates the calculated nearest-neighbor thermodynamic parameters; FIG. 4 shows the same for the experimental and predicted melting temperatures; FIG. 8, FIG. 10 and FIG. 12 show data for ZP 6 letter system, SB 6 letter system, and SBZP 8 letter. The first plot has an R2 correlation of 0.89; the second plot has an R2 of 0.87. On average, the Tm is predicted within 2.1° C. and the ΔG° 37 is predicted within 0.39 kcal/mol for the 94 GACTZPSB 8-letter DNA duplexes in this study (data in Tables 3, 6, and 8). These errors are similar to those observed for the nearest-neighbor parameters for standard DNA/DNA duplexes [M. M. Georgiadis, I., Singh, I., W. F. Kellett, S. Hoshika, S. A. Benner, N. G. J. Richards, J. Am. Chem. Soc. 137, 6947-6955 (2015)]. Thus, GACTZPSB 8-letter DNA reproduces, but in expanded form, the molecular recognition behavior of standard 4-letter DNA at the level of solution biophysics.

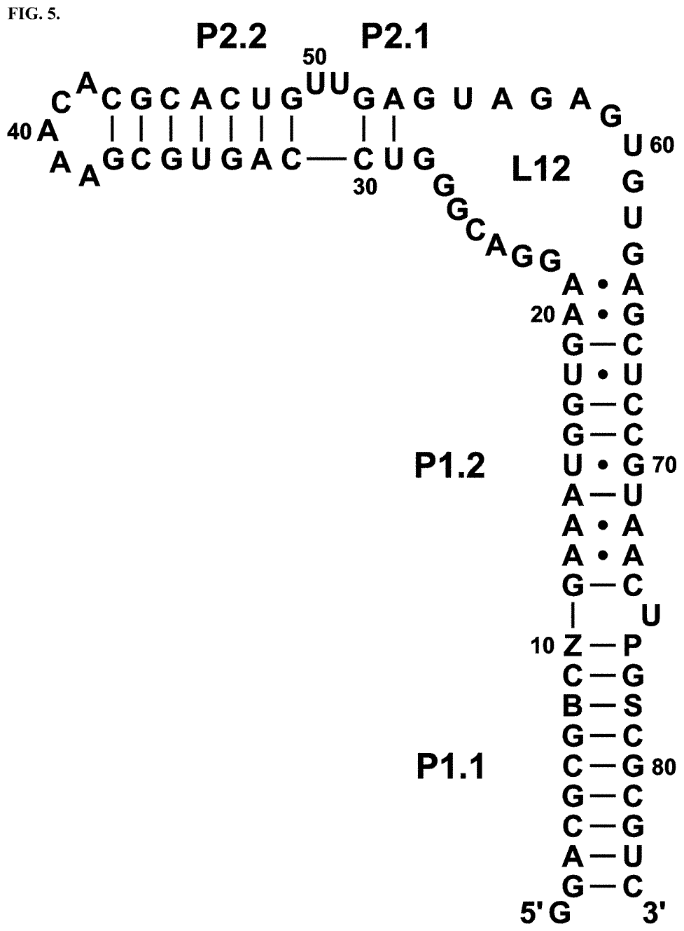

Experiments described in Example 2 establish that DNA oligonucleotides containing (in addition to A, T, G, and C heterocycles) heterocycles that implement the S, B, Z, and P hydrogen bonding patterns can direct, by transcription, the synthesis of RNA transcript products that have (in addition to A, U, G, and C heterocycles) heterocycles that implement the B, S, P and Z hydrogen bonding patterns. DNA oligonucleotides containing a promoter for the T7 RNA polymerase containing one or more non-standard nucleotides were synthesized. These included templates that contained only one non-standard nucleotide components. Further, a longer template was synthesized that encoded the “spinach” fluorescent aptamer [X. J. Lu, W. K. Olson, Nucleic Acids Res. 31, 5108-5121 (2003)], an RNA molecule 84 nucleotides in length that folds and binds the fluor 3,5-difluoro-4-hydroxybenzylidene imidazolinone. Upon binding, the fluor fluoresces green. One of the designed 8-letter RNA aptamers is shown schematically in FIG. 5.

### Procedures for the Transcription

To analyze the RNA transcripts, a set of analytical chemistry procedures were developed. These are described in Example 3. Central to these was “label shift” chemistry [J. S. Paige, K. Y. Wu, S. R. Jaffrey, Science 333, 642-646 (2011)], which was adapted to allow analysis of 8-letter RNA. Here, one of four standard RNA triphosphates is introduced into a transcription mixture with an alpha-³²P label. This leads to a product with a bridging ³²P-phosphate. Subsequent hydrolysis by ribonuclease T2 generates a mixture of nucleoside 3′-phosphates, where the 3′-nucleotide immediately preceding in the sequence carries a ³²P-label. The mixture of nucleoside 3′-phosphates is then resolved by chromatography to determine the adjacency patters of the system.

To identify useful RNA polymerases, initial studies were done with DNA templates containing only one nonstandard nucleotide in the 8-letter system. These studies showed that wild-type T7 RNA polymerase readily incorporated riboZTP opposite template dP, riboPTP opposite template dZ, and riboBTP opposite template dS. However, riboSTP was not incorporated opposite template dB. Without wishing to be bound by theory, this might be attributed to the absence of electron density delivered to the minor groove by the aminopyridone heterocycle on S. After substantial search, a T7 variant (H784A P266L Y639F) was discovered that was able to create RNA products that contain riboS, and RNA transcript products that contained all eight non-standard and standard nucleotides. This variant had been reported previously as able to accept modified 2′-ribose triphosphates without early termination or substantial infidelity, an unnatural structural difference different than the one proposed here [I. Hirao, T. Ohtsuki, T. Fujiwara, T. Mitsui, T. Yokogawa, T. Okuni, H. Nakagawa, K. Takio, T. Yabuki, T. Kigawa, K. Kodama, T. Yokogawa, K. Nishikawa, S. Yokoyama, Nature Biotechnol. 20, 177 (2002)]. Label shift experiments are described that specific incorporation of all four non-standard components of the 8-letter system into transcripts.

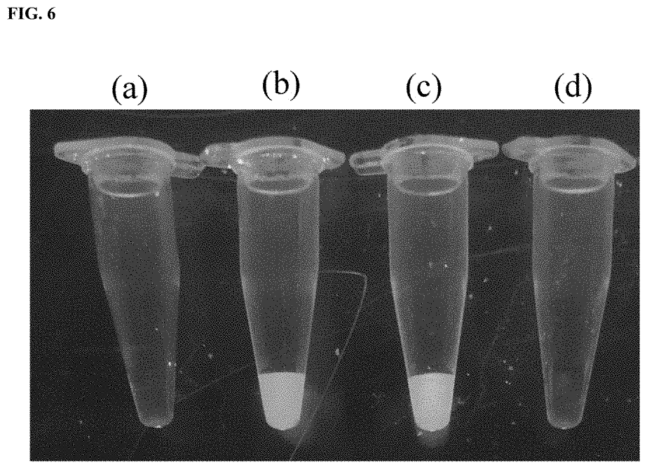

The full length 8-letter spinach variant was then prepared from the synthetic 8-letter DNA sequence placed behind a T7 promoter, isolated by gel electrophoresis, and studied. Notably, it fluoresced green when complexed to the fluor (FIG. 6). A number of variants of spinach lacking non-standard components of the 8-letter system were also prepared and studied. Of particular interest, placing 8-letter Z in the fold near the fluor quenched fluorescence, likely because Z's aminonitropyridone ring quenches fluorescence generally; analysis of the structure of native spinach suggested that the replacement did.

This result shows that the FAL variant of T7 RNA polymerase can incorporate riboSTP, notwithstanding the fact that the heterocycle on riboSTP does not have a moiety that delivers electron density to the minor groove. It is thus taught that the FAL variant will also incorporate riboKTP (in two forms, shown in FIG. 1), riboVTP (FIG. 1), and two forms of riboXTP (FIG. 1, but only the structures with Q).

In addition to allowing the synthesis by transcription of RNA molecules containing S, this invention makes available, also for the first time, an informational system that is built from eight different building blocks. This system has substantially increased information density; while a duplex with 10 nucleobase pairs built from a 4-letter alphabet has only 1,048,576 (=4¹⁰) different sequences, a duplex built from an 8-letter alphabet has 1,073,741,824 (=8¹⁰) different sequences. In terms of computer science bits, this doubles the information density of a DNA—like biopolymer. Further, detailed biophysical analysis of duplex suggests that the 8-letter molecular system has regular thermodynamic properties, just as four-letter DNA Such greater information storage capacity may have application in bar-coding and combinatorial tagging, computer retrievable information storage, and self-assembling nano-structures. Further, the fact that the number of letters in DNA can be doubled using a design theory that incorporates both hydrogen bonding and size complementarity increases confidence that the non-abridged Watson-Crick model reflects reality. Last, 8-letter DNA may now serve as a platform for more demanding goals in synthetic biology. One of these seeks to use the added information density to encode more amino acids in ribosome-based transcription.

What is claimed is:

1. A process for synthesizing RNA containing one or more non-standard nucleotides, wherein said process comprises contacting in aqueous solution
(a) a variant of T7 RNA polymerase that accept non-standard nucleoside triphosphates with
(b) a DNA template comprising a promoter for said variant, and
(c) nucleoside triphosphates that comprise one or more independently chosen heterocycles selected from the group consisting of 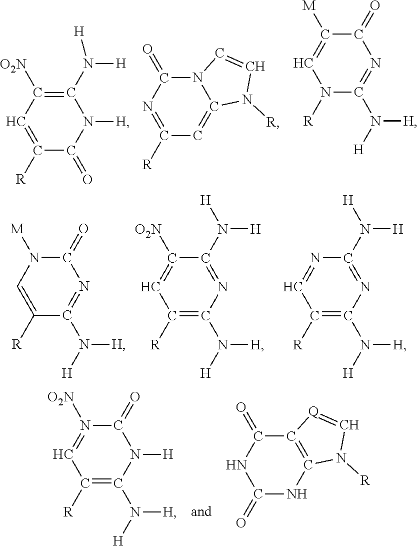
wherein Q is C—H, C-M or N, where M is an alkyl, alkenyl, or alkynyl substituent, either simple or functionalized, and R is the point of attachment of said heterocycle(s) to the ribose ring of said nucleoside triphosphate(s).

2. The process of claim 1, wherein said variant to T7 RNA polymerase has amino acids replaced at individual sites in its polypeptide sequence, wherein said replacements comprise a replacement of tyrosine at position 639 by phenylalanine, a replacement of histidine at position 784 by alanine, and a replacement of proline at position 266 by leucine.

3. The process of claim 1, wherein said nucleoside triphosphates comprise one or more independently selected heterocycles selected from the group consisting of 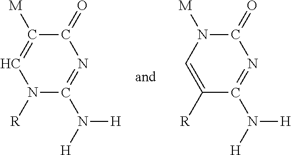
and M is an alkyl, alkenyl, or alkynyl substituent, either simple or functionalized, and R is the point of attachment of said heterocycle(s) to the ribose ring of said nucleoside triphosphate(s).

4. The process of claim 3, wherein M is methyl.

5. The process of claim 1, wherein said nucleoside triphosphates comprise one or more independently selected heterocycles selected from the group consisting of 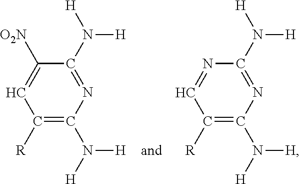
and R is the point of attachment of said heterocycle(s) to the ribose ring of said nucleoside triphosphate(s).

6. The process of claim 1, wherein said nucleoside triphosphate(s) comprise(s) the heterocycle 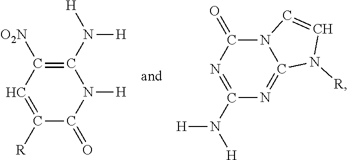
wherein R is the point of attachment of said heterocycle(s) to the ribose ring of said nucleoside triphosphate(s).

7. The process of claim 1, wherein said nucleoside triphosphate(s) comprise(s) the heterocycle 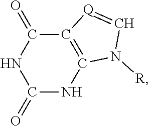
wherein Q is C—H, C-M or N, where M is an alkyl, alkenyl, or alkynyl substituent, either simple or functionalized, and R is the point of attachment of said heterocycle(s) to the ribose ring of said nucleoside triphosphate(s).

8. A composition of matter, said composition being a molecule that is an analog of RNA, wherein
(a) one or more of the nucleotides in said molecule has, instead of adenine, uridine, cytosine, or guanine, the heterocycle 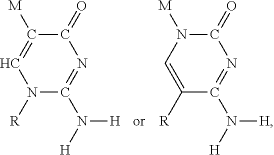
(b) one or more of the nucleotides in said molecule has, instead of adenine, uridine, cytosine, or guanine, the heterocycle 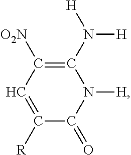
(c) one or more of the nucleotides in said molecule has, instead of adenine, uridine, cytosine, or guanine, the heterocycle 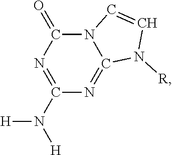
and
(d) one or more of the nucleotides in said molecule has, instead of adenine, uridine, cytosine, or guanine, the heterocycle 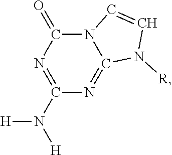
wherein R is the point of attachment of said heterocycle(s) to the ribose ring of said nucleoside triphosphate(s).
